(19) United States
(12) Patent Application Publication
(10) Pub. No.: US 2012/0253047 A1
(43) Pub. Date: Oct. 4, 2012

(54) PROCESS FOR THE PREPARATION OF (R)-PRAMIPEXOLE

(75) Inventors: Pietro ALLEGRINI, San Donato Milanese (IT); Gabriele Razzetti, Sesto San Giovanni (IT); Vittorio Lucchini, San Donato Milanese (IT)

(73) Assignee: Dipharma S.p.A., Mereto Di Tomba (IT)

(21) Appl. No.: 13/450,803

(22) Filed: Apr. 19, 2012

Related U.S. Application Data
(63) Continuation-in-part of application No. 12/776,588, filed on May 10, 2010, now Pat. No. 8,203,002.
Application No. 12/776,588 is a division of application No. 10/593,269, filed on Sep. 18, 2006, now Pat. No. 7,741,490.

(30) Foreign Application Priority Data
Mar. 19, 2004 (IT) MI2004A000531
Jan. 28, 2005 (IT) MI2005A000126

(51) Int. Cl. C07D 277/82

(52) U.S. Cl. 548/164

(57) ABSTRACT

The invention relates to a novel process for the preparation of (R)-2-amino-6-n-propylamino-4,5,6,7-tetrahydrobenzothiazole, or a salt thereof.

This application is a Continuation in Part of application U.S. Ser. No. 12/776,588, filed on May 10, 2010, which is a Divisional application of U.S. Ser. No. 10/593,269, filed on Sep. 18, 2006, now U.S. Pat. No. 7,741,490 which issued Jun. 22, 2010, which is a 35 U.S.C. 371 National Phase Entry Application from PCT/EP2005/002641, filed Mar. 11, 2005, which claims the benefit of Italian Patent Application No. MI2004A000531 filed on Mar. 19, 2004 and Italian Patent Application No. MI2005A000126 filed on Jan. 28, 2005, the disclosures of which are incorporated herein in their entirety by reference.

### FIELD OF THE INVENTION

The present invention relates to a novel process for the preparation of (R)-2-amino-6-n-propylamino-4,5,6,7-tetrahydrobenzothiazole, or a salt thereof.

### TECHNOLOGICAL BACKGROUND

(R)-2-amino-6-n-propylamino-4,5,6,7-tetrahydrobenzothiazole dihydrochloride of formula (A), is known from U.S. Pat. No. 4,843,086. EP 1453505 discloses its utility in treating chronic neurodegenerative diseases, for instance amyotrophic lateral sclerosis (ALS).

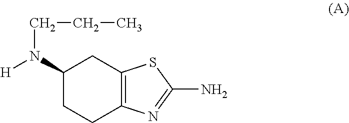

U.S. Pat. No. 6,727,367 discloses inter alia a method for the resolution or the enrichment of (R,S)-2-amino-6-propylamino-4,5,6,7-tetrahydrobenzothiazole in the single (R) or (S) enantiomers, in particular in the (S) enantiomer. U.S. Pat. No. 7,662,610 discloses an enzymatic method for the preparation of a derivative of the such single (R) or (S) enantiomer, and the use of the (S) enantiomer for preparing Pramipexole. Synthetic routes known for the preparation of Pramipexole are also described in U.S. Pat. No. 4,886,812, EP 186087, EP 207696 and J. Med. Chem. 30. 494 (1987).

### SUMMARY OF THE INVENTION

In a first aspect the invention provides a novel a process for the preparation of (R)-2-amino-6-n-propylamino-4,5,6,7-tetrahydrobenzothiazole of formula (I), as herein defined, or a salt thereof, which employs novel intermediates and fulfils the requirements for its in industrial production.

In a second aspect the invention provides compèound (R)-2-amino-6-n-propylamino-4,5,6,7-tetrahydrobenzothiazole free base in crystalline form, in particular in the crystalline form hereinafter referred to as Form A, and a method for its preparation.

In a third aspect the invention provides compound (R)-2-amino-6-n-propylamino-4,5,6,7-tetrahydrobenzothiazole monohydrate dihydrocloride salt in crystalline form, in particular in the crystalline form hereinafter referred to as Form B, and a method for its preparation.

Furthermore, the invention provides a pharmaceutical composition containing (R)-2-amino-6-n-propylamino-4,5,6,7-tetrahydrobenzothiazole in crystalline form, in particular the form designated as Form A, or a salt thereof, in particular the monohydrate dihydrocloride salt thereof, designated as Form B, or a mixture thereof as active ingredient in admixture with an excipient and/or carrier, and its use in therapy.

### DESCRIPTION OF THE FIGURES

The novel crystalline Forms A and B were characterised by X-ray powder diffraction (XRPD), ¹H-NMR and ¹³C-NMR Nuclear Magnetic Resonance spectrometry, and infra-red spectrophotometry (FT-IR). The water content of the compounds was determined by titration with the Karl Fischer technique. The X-ray diffraction spectra (XRPD) were collected with the APD-2000 automatic powder and liquid diffractometer, manufactured by Ital-Structures, under the following operating conditions: Bragg-Brentano geometry, CuKα radiation (λ=1,5418 Å), scanning with a 2θ angle range of 3-40° and a step size of 0.03°, for a time of 1 sec. The ¹H-NMR and ¹³C-NMR spectra were acquired with a Varian Mercury 300 spectrometer, using DMSO-d6 as solvent. The IR spectra were recorded with a Perkin-Elmer Paragon 500 spectrophotometer for 16 scans between 4000 and 650 cm⁻¹, Particle size was determined with the known laser light scattering technique using a Malvern Mastersizer MS1 instrumentation under the following operative conditions:
300RF mm lens, with 2.4 mm laser beam length; and
500 mg sample dispersed in 10 ml of hexane (ACS reagent) with
1% SPAN 85®, no presonication, 2500 rpm stirring rate.

FIG. 1. XRPD spectrum (X-ray powder diffraction) of a compound of formula (I) in a crystalline form designated as Form A.

FIG. 2. XRPD spectrum of a compound of formula (I) as monohydrate dihydrocloride salt in a crystalline form designated as form B.

FIG. 3. IR spectrum of a compound of formula (I) as monohydrate dihydrocloride salt in a crystalline form designated as form B.

### DETAILED DISCLOSURE OF THE INVENTION

The present invention relates to a process for the preparation of a compound of formula (I), in a solid state thereof, or a salt thereof,

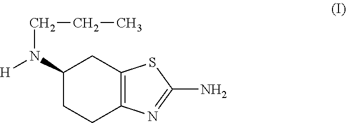

comprising:
a) obtaining a compound of formula (II), as single (R) enantiomer, or a salt thereof,

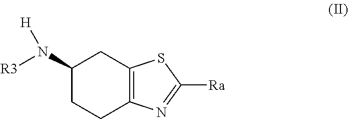

wherein Ra is a protected amino group and R₃ is hydrogen or a R₄—O—CO— group, wherein R₄ is a straight or branched C₁-C₄ alkyl; by rearrangement of a compound of formula (III), or a salt thereof, as single (R) enantiomer,

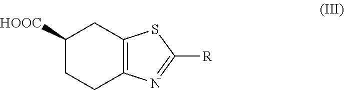

wherein R is a protected amino group; via formation of isocyanate, and subsequent addition of a nucleophilic solvent or subsequent quenching in water in the presence of an acid; or
b) obtaining compound of formula (II), as single (R) enantiomer, or a salt thereof,

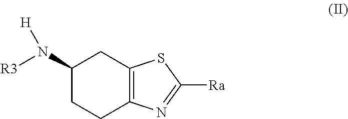

wherein Ra is a free amino group and R₃ is hydrogen; by rearrangement of a compound of formula (III), or a salt thereof, as single (R) enantiomer, as defined above; via formation of isocyanate, and subsequent addition of water, to obtain a compound of formula (IV)

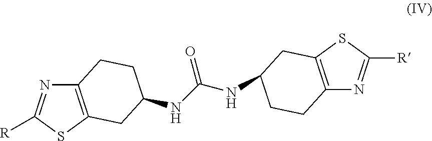

wherein R′ has the same meaning as R above, and subsequent hydrolysis;

alkylating a compound of formula (II), thus obtained, to obtain a compound of formula (V)

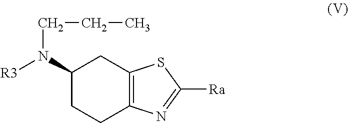

wherein R₃ and Ra are as defined above; and, if necessary, removing the primary amino-protecting group and/or the R₄—O—CO— group from the secondary amino group, to obtain a compound of formula (I), and, if desired, converting a compound of formula (I) into a salt thereof, and/or converting a salt of a compound of formula (I) into the free compound.

A solid state of a compound of formula (I), or a salt thereof, is in particular the amorphous or an anhydrous or hydrate, preferably a monohydrate, crystalline form thereof.

It will be appreciated that a compound of formula (V), wherein Ra is a free amino group and R₃ is hydrogen, is a compound of formula (I). In particular said compound of formula (I) as monohydrate dihydrocloride salt is herein defined also as (R)-Pramipexole or Dexpramipexole.

According to the present invention, a protected amino group R or Ra can be, for example, a protected amino group in the form of an acylamino, carbamoyl, arylmethylamino, phthalimido or silylamino group.

In an acylamino group, acyl is for example formyl or C₁-C₆—CO— alkyl, preferably acetyl, propionyl or pivaloyl, optionally substituted with 1 to 3 halogen atoms, such as chlorine, fluorine, bromine or iodine.

In a carbamoyl group, the amino group is linked, for example, to a C₁-C₆ alkoxy-carbonyl group, wherein the alkyl residue is straight or branched, optionally substituted with phenyl. The alkyl residue is preferably a C₁-C₄ alkyl group, optionally substituted with phenyl, such as methyl, ethyl, n-propyl, isopropyl, n-butyl, isobutyl, tert-butyl, benzyl or phenylethyl, in particular tert-butyl.

In an arylmethylamino group, for example a mono, di- or particularly tri-arylmethylamino group, the aryl moiety is preferably a phenyl group. Said group is for example benzyl-, diphenylmethyl- or trityl-amino; more particularly a 1-methyl-1-phenyl-ethylamino group.

A silylamino group is for example a trimethylsilylamino or tert-butyl-dimethylsilylamino group.

A protected amino group R or Ra is preferably a protected amino group such as an acylamino or arylmethylamino group, in particular acylamino, wherein acyl is formyl, acetyl, propionyl or pivaloyl, the latter three being optionally substituted with 1 to 3 halogen atoms, such as chlorine, fluorine, bromine or iodine. More preferably the R group is acetylamino, propionylamino or pivaloylamino.

A salt of a compound of formula (I), (II) or (V) is preferably a pharmaceutically acceptable salt thereof, and can be obtained according to known methods. In particular a salt of a compound of formula (I) is preferably the monohydrochloride or dihydrochloride salt.

A salt of a compound of formula (III) can be a salt with a organic or inorganic base or a organic or inorganic acid. Preferred examples of salts with a base are those with an inorganic base, such as sodium, lithium or potassium salts, or salts with a primary, secondary or tertiary amine, such as N-methyl-, N,N-dimethyl- and triethyl-ammonium salts, benzylammonium, α-methylbenzylamine, N-methyl-D-glucamine, cinchonidine or cinchonine salts. Preferred examples of salts with acids are those with hydrochloric, sulfuric, acetic, oxalic or methanesulfonic acids, preferably with an optically active acid, such as tartaric or camphorsulfonic acid.

Preferably a compound of formula (III), as single (R)-enantiomer has, has an enantiomeric purity typically at least about 96%, more preferably at least about 99%.

Preferred examples of the compounds of formula (III) are: (R)-2-acetylamino-4,5,6,7-tetrahydro-benzothiazole-6-carboxylic acid; and (R)-2-propionylamino-4,5,6,7-tetrahydro-benzothiazole-6-carboxylic acid, or a salt thereof.

According to process variant a), a nucleophilic solvent can be for example a C₁-C₄ alkanol, typically methanol, ethanol or i-propanol.

An acid is for example a mineral or an organic acid, in particular hydrochloric, sulfuric, formic or acetic acid.

According to process variants a) and b) rearrangement of a compound of formula (III) can be effected for example according to the Schmidt, Lossen, Hofmann or Curtius reactions.

The sequence of the products formed during the rearrangement reaction is the following:

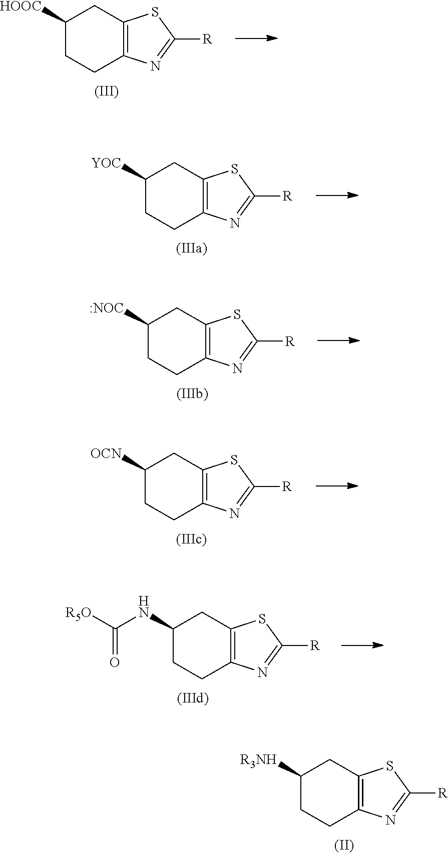

in which Y is NHOCOR₄, N₃ or NH₂, wherein R₄ is as defined above; R₅ is hydrogen or straight or branched C₁-C₄ alkyl; and R and R₃ are as defined above.

All of the Schmidt, Lossen, Hofmann and Curtius reactions make use of an isocyanate of formula (IIIc) as defined above.

A compound of formula (IIIc) can be prepared according to the Schmidt reaction, by treating a compound of formula (III) with hydrazoic acid in the presence of sulfuric acid to obtain a compound of formula (IIIa), wherein Y is N₃ and R is as defined above, which is converted to the corresponding compound of formula (IIIc) by heating.

Alternatively, a compound of formula (IIIc) can prepared according to the Lossen reaction, by reaction of a compound of formula (III) with a halogenating agent, preferably thionyl chloride or oxalyl chloride, and subsequent reaction with an acyl-hydroxylamine, preferably acetyl-hydroxylamine, thereby obtaining the respective acylated hydroxamic acid, i.e. a compound of formula (IIIa) wherein Y is NHOCOR₄ and R is as defined above. Treatment of the latter with an alkali hydroxide affords a compound of formula (IIIc).

Again, a compound of formula (IIIc) can be prepared according to the Hofmann reaction, by transforming the carboxylic acid into amide according to known methods, i.e. into a compound of formula (IIIa) wherein Y is NH₂ and R is as defined above, then treating it with an alkali hypohalogenite, preferably sodium hypochlorite.

Finally, a compound of formula (IIIc) can be prepared according to the Curtius reaction, by reaction of a compound of formula (III) with a halogenating agent, preferably thionyl chloride or oxalyl chloride, and subsequent treatment with sodium azide to obtain the respective acyl-azide of formula (IIIa) wherein Y is N₃ and R is as defined above; or directly with diphenylphosphorylazide, in the presence of an organic base, in particular triethylamine, diisopropylethylamine or pyridine. The acyl-azide of formula (IIIa) is converted to the corresponding compound of formula (IIIc) by heating.

The rearrangement reactions reported above can be carried out according to known methods, for example at a temperature approximately ranging from about 10° C. to the reflux temperature, for a time ranging between 2 hours and 15 hours, preferably between 5 hours and 10 hours.

More particularly, a compound of formula (IIIa), in which Y is N₃, is poured in water in the presence of an acidic agent, thereby converting it to a compound of formula (IIId) as defined above. An acidic agent is for example a mineral or organic acid, in particular hydrochloric, sulfuric, formic or acetic acid, in amounts ranging from about 2 to about 5 mols, preferably from about 2.5 to about 3.5 mols. The reaction is carried out at a temperature ranging from room temperature to the reaction mixture reflux, preferably from about 50 to about 80° C. When the nucleophilic solvent is for example water, in the resulting compound of formula (IIId) R₅ is hydrogen. Alternatively, when the nucleophilic solvent is for example a C₁-C₄ alkanol, in particular methanol, ethanol or i-propanol, in the resulting compound of formula (IIId) R₅ is alkyl.

According to a preferred aspect, the rearrangement reaction to form the acyl-azide of formula (IIIa) in which Y is N₃ is carried out according to Curtius in a nucleophilic solvent, as defined above. The reaction proceeds until formation of a compound of formula (IIId) wherein R₅ is a straight or branched C₁-C₄ alkyl group, with no need for isolating any intermediate.

A compound of formula (IIId) in which R₅ is hydrogen spontaneously transforms into a compound of formula (II), wherein Ra is a protected amino group and R₃ is hydrogen. A compound of formula (IIId) in which R₅ is alkyl is a compound of formula (II) wherein R₃ is a R₄—O—CO— group, as defined above and Ra is a protected amino group.

Alternatively, when a compound of formula (IIIa), in which Y is N₃, is poured in water, or vice versa, a compound of formula (IV)

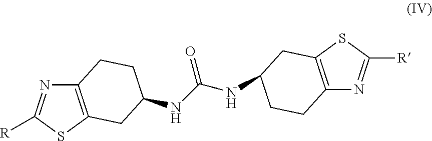

wherein R and R′ are as defined above is obtained, which is hydrolyzed to a compound of formula (II) wherein Ra is a free amino group and R₃ is hydrogen. The hydrolysis is typically an acidic hydrolysis, for example by treatment with hydrochloric acid according to known methods.

The alkylation of a compound of formula (II) and, if the case, the removal of the primary amino protecting group and, if present, of the R₄—O—CO— group from the secondary amino group present in a compound of formula (V), can be carried out according to U.S. Pat. No. 4,843,086.

According to a preferred aspect of the invention, alkylation a compound of formula (II), wherein R₃ is hydrogen and Ra is as defined above, comprises acylation of said compound of formula (II) by reaction with propionic anhydride, and subsequent reduction of the compound of formula (VI) thus obtained,

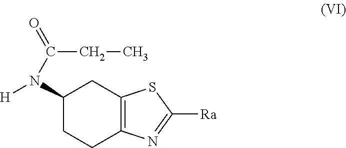

wherein Ra is as defined above, by treatment with an alkali metal borohydride and molecular iodine, to obtain a compound of formula (V), as defined above, wherein R₃ is hydrogen and Ra is as defined above; followed, if necessary, by deprotection of the primary amino group and, if the case, the conversion of a compound of formula (VI) into a salt thereof, and/or conversion of a salt of a compound of formula (VI) into the free compound.

The acylation of a compound of formula (II) with propionic anhydride can be carried out according to known methods.

An alkali metal borohydride is for example NaBH₄ or KBH₄, preferably NaBH₄. The amount of alkali metal borohydride used in the reduction, for example NaBH₄, is about 1-5 mols per mole of compound of formula (VI), preferably from about 2 to 4 mols, whereas the molar amount of iodine is about 0.5-3 mols per mole of compound of formula (VI), preferably from about 1 to 2. The reduction of a compound of formula (VI) is preferably carried out in an ether solvent, such as tetrahydrofuran, dioxane or diethyl ether, in particular tetrahydrofuran. The reaction can be carried out at a temperature ranging from about 0° C. to the reflux temperature, preferably at approx. 20-40° C.

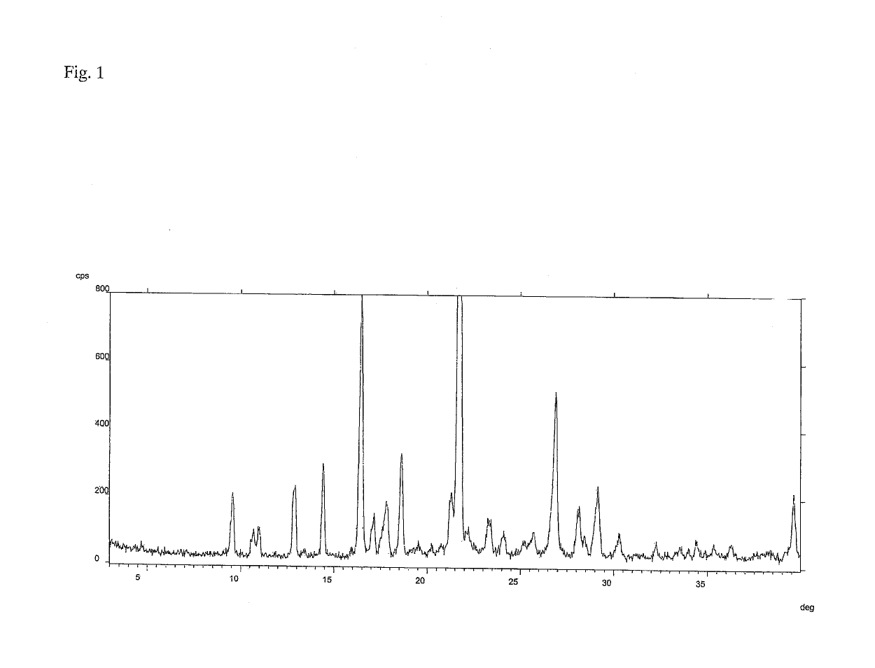

According to a further feature the invention provides a compound of formula (I), as defined above, as free base, namely (R)-2-amino-6-n-propylamino-4,5,6,7-tetrahydrobenzothiazole free base, in crystalline form, in particular in a crystalline form herein designated as Form A, characterized by an XRPD spectrum as shown in FIG. 1, wherein the most intense peaks fall at 9.66, 10.77, 11.10, 12.99, 14.55, 16.56, 17.19, 17.94, 18.72, 21.42, 21.84, 23.49, 24.21, 27.00 and 28.23±0.2° in 2θ, and a method for its preparation.

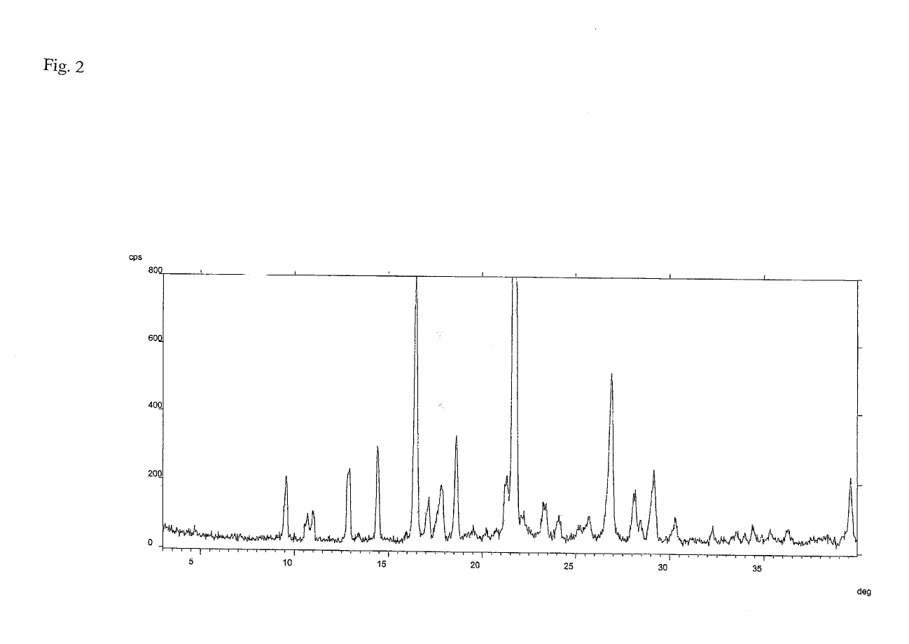

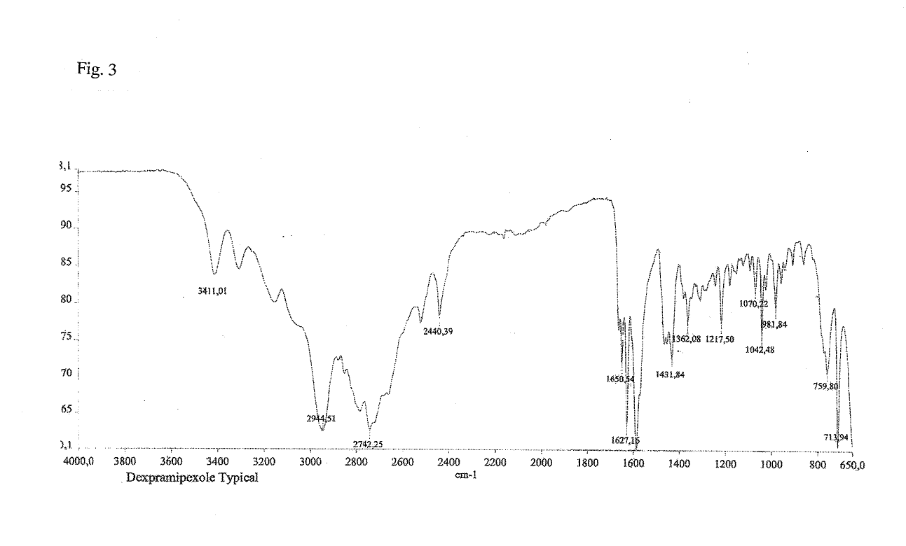

According to a further aspect the invention provides the hydrochloride salt of a compound of formula (I), as defined above, namely (R)-2-amino-6-n-propylamino-4,5,6,7-tetrahydrobenzothiazole monohydrate dihydrocloride salt, in crystalline form. In particular in a crystalline form herein designated as Form B, characterised by an XRPD spectrum as shown in FIG. 2, wherein the most intense peaks fall at 6.48, 12.06, 12.93, 13.29, 13.92, 15.15, 16.95, 17.25, 17.91, 19.47, 21.36, 24.21, 24.75, 24.99, 25.59 and 28.44±0.2° in 2θ; and an IR spectrum as shown in FIG. 3, showing the most intense peaks at 3411, 2945, 2742, 2440, 1651, 1627, 1432, 1362, 1218, 1070, 1042, 982, 760 e 714 cm-1. Said crystalline form has a water content ranging between about 5 and about 7% w/w, typically around 6% w/w, so as to be defined as monohydrate.

A compound of formula (I) as defined above, as a free base, in particular as crystalline Form A, has a high purity degree, typically ≧99.5% HPLC; and its monohydrate dihydrocloride salt, in particular as crystalline Form B, is endowed with a very high purity degree, typically ≧99.95% HPLC.

The size of the crystals of (R)-2-amino-6-n-propylamino-4,5,6,7-tetrahydrobenzothiazole free base Form A, and in particular of monohydrate dihydrocloride salt, Form B, as obtainable according to the invention, is characterized by a D₅₀ value ranging from about 25 to 250 μm. If desired, said value can be reduced by micronisation or fine grinding.

A compound of formula (III), or a salt thereof, as defined above, as single (R) enantiomer, can be obtained by a process comprising the hydrolysis of an ester of formula (VII), or a salt thereof, either as a mixture of (R,S) enantiomers or as single (R) or (S) enantiomer

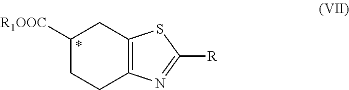

wherein R is a protected amino group; R₁ is straight or branched C₁-C₆ alkyl, optionally substituted with phenyl; and the asterisk * indicates the stereogenic carbon atom; and, if the case, the resolution of the mixture of (R,S) enantiomers of the compound of formula (III) to yield the single (R) enantiomer, and/or the conversion of the single (S) enantiomers of the compound of formula (III) into the single (R) enantiomer by racemization followed by resolution.

R₁ is preferably a C₁-C₄ alkyl group, such as methyl, ethyl, n-propyl, isopropyl, n-butyl, isobutyl or tert-butyl, in particular ethyl or propyl; or benzyl or phenylethyl.

A salt of a compound of formula (VI) or (VII) is for example a salt with a mineral acid, preferably an hydrohalic acid, in particular hydrochloric or hydrobromic acid, or an organic acid, such as acetic, oxalic or methanesulfonic acid, preferably an optically active acid, such as tartaric or camphorsulfonic acid.

The hydrolysis of a compound of formula (VII) can be carried out by reaction with an alkali hydroxide, for example sodium or potassium hydroxide, in amounts from about 1 to 4 equivalents, preferably from 1.5 to 2.5 equivalents, in a polar protic solvent, for example water or C₁-C₄ alkanols, in particular methanol, ethanol, i-propanol, or mixtures thereof; at a temperature ranging from about 0° C. to the solvent reflux, preferably from about 10 to 50° C., in particular at approx. 20° C.

According to the invention, a mixture of (R,S) enantiomers can contain the two single enantiomers in any ratio to each other. The enantiomeric purity is generally expressed as “enantiomeric excess” and defined, for example, for the (R) enantiomer as (R−S)/(R+S)×100 wherein S and R are respectively the amounts of the (S) and (R) enantiomers. According to the invention, the expression single (R) enantiomer means that the enantiomeric purity is usually at least about 99.5%, preferably at least about 99.9%, most preferably equal to or higher than 99.95%.

The optional resolution of the mixture of (R,S) enantiomers of a compound of formula (III) into the single (R) enantiomers can be carried out, for example, by fractional crystallization of the diastereomeric salts of a compound of formula (III) obtained by reaction with optically active, enantiomerically pure acids or bases. An example is the reaction of the compound of formula (III) with an enantiomerically pure aliphatic or aromatic amine, for example α-methylbenzylamine, N-methyl-D-glucamine, cinchonidine and cinchonine; or with an enantiomerically pure acid, for example tartaric acid or camphorsulfonic acid, in a solvent capable of promoting the formation of the salt and the subsequent precipitation of the desired diastereomer. Examples of said solvents are C₁-C₄ alkanols, such as methanol, ethanol and i-propanol; ketones, such as acetone; ethers such as tetrahydrofuran and dioxane; alkyl esters, such as ethyl acetate; amides, such as dimethylformamide and dimethylacetamide; dimethylsulfoxide; or mixtures thereof or mixtures of one or more of them with water. The temperature can range from room temperature to the solvent reflux temperature. Alternatively, the resolution can be carried out by means of preparative chromatography using a chiral, optically active stationary phase, including the “Simulating Moving Bed” (SMB) technology. A further alternative can consists in the enzymatic resolution, either by selective hydrolysis of one stereoisomer of an ester of formula (VII) to an acid of formula (III), or by selective esterification of one stereoisomer of an acid of formula (III) to an ester of formula (VII). A compound of formula (III), or a salt thereof, as defined above, as single (R) enantiomer, is preferably obtained according to the process disclosed by U.S. Pat. No. 7,662,610.

U.S. Pat. No. 4,988,699 discloses compounds of formula (III) and of formula (VII) as (R,S) mixtures in which the R substituent is an amino group optionally substituted with various groups, inter alia lower alkanoyl groups.

A compound of formula (VII), and the salts thereof, can be obtained by a process comprising converting the amino group of a compound of formula (VIII)

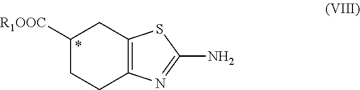

wherein R₁ and the asterisk * have the meanings reported above, into a protected amino group R as defined above, and the optional resolution of the mixture of (R,S) enantiomers of a resulting compound of formula (VII) into the single (R) enantiomer thereof, and/or salification thereof.

The conversion of the amino group of a compound of formula (VII) to a protected amino group R, preferably in an acylamino, carbamoyl, arylmethylamino, phthalimido or silylamino group, as well as the salification, can be carried out according to known methods. The protection as an acylamino or carbamoyl group is preferably carried out by reaction with the corresponding anhydride, in particular acetic anhydride, or acyl-chloride or alkoxycarbonyl-chloride, in particular acetyl-chloride or methoxy- or ethoxy-carbonyl-chloride, in a solvent selected for example from acetone, acetonitrile, tetrahydrofuran, dioxane, dichloromethane or toluene; in the presence of a basic agent, preferably triethylamine, diisopropylamine or pyridine. The reaction is carried out from about −15° C. to the solvent reflux, preferably between about 0° C. and 50° C., in particular at room temperature. The optional resolution of a mixture of (R,S) enantiomers of a compound of formula (VII) into the single (R) enantiomer can be obtained, for example, by reaction with an organic acid, according to the procedures reported above for the resolution of a mixture of (R,S) enantiomers of a compound of formula (III). A compound of formula (VIII) can be prepared by reaction of a compound of formula (IX),

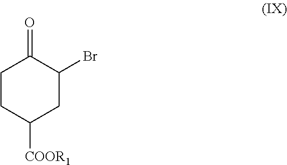

wherein R₁ is as defined above, with thiourea. The cyclization reaction is carried out in an organic solvent, for example a C₁-C₄ alkanol, acetone, tetrahydrofuran, dioxane or mixtures thereof, at a temperature ranging from about 0° C. to the solvent reflux temperature, for a time ranging between 1 hour and 8 hours, in particular between 2 hours and 5 hours. The hydrobromide salt of a compound of formula (VIII) forms first and is then converted to the free base form by suspending it for example in water, C₁-C₆ alkanols or acetone, preferably methanol or ethanol; at a temperature ranging from room temperature to the solvent reflux temperature; and adding from 1 to 1.5 equivalents, preferably from 1 to 1.1 equivalents, of an inorganic base, preferably sodium or potassium bicarbonate. Upon filtration, a compound of formula (VIII) separates as the free base.

In particular, a compound of formula (VII), as defined above, wherein the protected amino group R is in the form of an acylamino or carbamoyl group, can be prepared by reaction of a compound of formula (IX), as defined above, with a compound of formula (X)

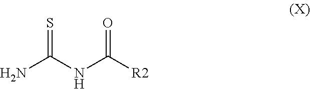

wherein R₂ is respectively a straight or branched C₁-C₆ alkyl or alkoxy group, optionally substituted with phenyl.

R₂ is preferably a C₁-C₄ alkyl group, optionally substituted with phenyl, such as methyl, ethyl, n-propyl, isopropyl, n-butyl, isobutyl, tert-butyl, benzyl or phenylethyl, in particular methyl. Alternatively, it is preferably a C₁-C₄ alkoxy group, optionally substituted with phenyl, for example methoxy, ethoxy, propoxy or benzyloxy, in particular methoxy.

The hydrobromide salt of a compound of formula (VII) is first obtained, which is then converted to the free base form.

The reaction between a compound of formula (IX) and a compound of formula (X) can be carried out according to the above reported procedure by reaction between a compound of formula (IX) and thiourea. The hydrobromide salt of a compound of formula (VII) can be converted to the free base form according to the procedure reported above for the transformation of a hydrobromide salt of a compound of formula (VIII) to the free base form.

The compounds of formula (IX) and (X) can be prepared with known methods. For example, a compound of formula (IX) can be prepared by monobromination of the corresponding ketone of formula (XI)

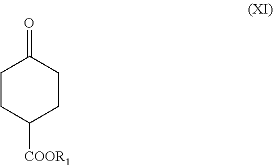

wherein R₁ is as defined above, with 0.8-1.5 equivalents, preferably 1 equivalent, of bromine in a solvent selected for example from dichloromethane, toluene, acetic acid or a C₁-C₄ alkanol, in the presence of hydrobromic acid in amounts approx. ranging from 0 to 0.2 equivalents. The reaction is carried out at a temperature ranging from about −15° C. to 40° C., preferably from 0° C. to 15° C., for a time ranging between 1 hour and 6 hours, preferably between 2 hours and 5 hours. A compound of formula (XI) is commercially available.

The process of the invention is particularly advantageous for the production on an industrial scale of a compound of formula (I), or a salt thereof, in particular as monohydrate dihydrocloride salt, typically in crystalline form, and preferably in the crystalline form herein designated as Form B. In fact, the resolution of the enantiomers takes preferably place during the first synthetic steps and moreover the discarded enantiomer can be recovered by racemization and recycled. This attains a reduction in the by-products of the more expensive final products and higher yields.

Accordingly the compound (R)-2-amino-6-n-propylamino-4,5,6,7-tetrahydrobenzothiazole monohydrate dihydrocloride salt in crystalline form, in particular in the crystalline form herein designated as Form B, shows an enantiomeric purity of at least about 99.5%, preferably at least about 99.9%, most preferably equal or higher than 99.95%.

According to a further aspect the invention provides a pharmaceutical composition, preferably for oral administration, containing a therapeutically effective amount of a compound formula (I) as defined above in crystalline form, preferably in crystalline Form A, or a salt thereof, in particular the monohydrate dihydrochloride salt thereof in crystalline form, preferably in crystalline Form B, or a mixture thereof, as active ingredient in admixture with an excipient and/or carrier, and its use in therapy.

Said pharmaceutical compositions can be prepared according to known methods in pharmaceutical technique, and can be used for treating and restoring neuronal, smooth and striated muscular and/or retinal tissue function in children and adults afflicted with chronic neurodegenerative diseases, such as neurodegenerative movement disorders and ataxias, seizure disorders, motor neuron disorders and inflammatory demyelinating disorders. Examples of disorders include Alzheimer's disease, Parkinson's disease and amyotrophic lateral sclerosis.

As used herein, the term “treating” includes alleviation of the symptoms associated with a specific disorder or condition and/or preventing or eliminating said symptoms.

The dosage of a compound of formula (I) in crystalline form, in particular Form A, or a salt thereof, in particular the monohydrate dihydrochloride salt thereof in crystalline form, in particular Form B, or a mixture thereof, to be orally administered to mammals, including humans, may range from about 0.1 to about 300 mg/Kg/daily, preferably from about 0.5 to about 50 mg/Kg/daily. For treating humans with acute brain injury a single dose between about 10 mg and about 100 mg can be parenterally administered, or by continuos intravenous infusion between about 10 mg/day and about 500 mg/day. Anyway, the more suitable dosage and route of administration is left to the skill of the physician.

The following examples illustrate the invention.

### Example 1

### 2-Amino-4,5,6,7-tetrahydro-benzothiazole-6-carboxylic acid ethyl ester hydrobromide [(VIII) R₁=ethyl]

A 3 liter reactor equipped with mechanical stirrer, thermometer and condenser was loaded with 1500 ml of ethanol and 200 g of 4-oxo-cyclohexanecarboxylic acid ethyl ester. After cooling to 0° C., 188 g of bromine were dropped therein in about 1 hour. The temperature was raised to 10° C., then to the room one after discolouration. After 1 hour, 89.32 g of thiourea were added in portions to obtain a suspension, that was refluxed to obtain gradual dissolution of the solid. After 4 hours the solution was concentrated to small volume to obtain a solid mass, that was suspended in 800 ml of acetone and refluxed to obtain a solution. The solution was then cooled to room temperature to precipitate a solid, then to 0° C. and after 4 hours the solid was filtered, washed twice with 100 ml of cold acetone and dried to obtain 170 g of the title product.

¹H-NMR in DMSO: 1.20 ppm (t, 3H); 1.79 ppm (m, 1H); 2.05 ppm (m, 1H); 2.43 ppm (t, 2H); 2.70 ppm (m, 3H); 4.08 ppm (q, 2H); 6.63 ppm (s, 2H).

### Example 2

### 2-Amino-4,5,6,7-tetrahydro-benzothiazole-6-carboxylic acid ethyl ester [(VIII) R₁=ethyl]

A 2 liter reactor equipped with mechanical stirrer, thermometer and condenser was loaded with 600 ml of water, 110 g of 2-amino-4,5,6,7-tetrahydro-benzothiazole-6-carboxylic acid ethyl ester hydrobromide [(III), R₁=ethyl] and 120 ml of methanol. The mixture was refluxed and hot filtered on a Celite bed. The resulting solution was added with a solution of 32 g of sodium bicarbonate in 300 ml of water (final pH=7-8). After 2 hours at room temperature, the precipitated white solid was filtered, washed with 100 ml of water and dried to obtain 72 g of the title product.

¹H-NMR in DMSO: 1.20 ppm (t, 3H); 1.79 ppm (m, 1H); 2.05 ppm (m, 1H); 2.43 ppm (t, 2H); 2.70 ppm (m, 3H); 4.08 ppm (q, 2H); 6.63 ppm (s, 2H).

### Example 3

### 2-Acetylamino-4,5,6,7-tetrahydro-benzothiazole-6-carboxylic acid ethyl ester [(VII), R₁=ethyl, R=—NH—CO—CH₃]

A 500 ml reactor equipped with mechanical stirrer, thermometer and condenser was loaded with 280 ml of acetonitrile, 71 g of 2-amino-4,5,6,7-tetrahydro-benzothiazole-6-carboxylic acid ethyl ester [(VIII), R₁=ethyl] and 38.75 g of acetic anhydride. 38.03 g of triethylamine were dropwise added to the resulting suspension in about 10 minutes. The suspension was refluxed, obtaining complete dissolution at a temperature ranging from 70 to 75° C. After approx. 2 hours 30 minutes the solution was concentrated to dryness, and the residue was crystallized from 450 ml of isopropanol to obtain 74.5 g of 2-acetylamino-4,5,6,7-tetrahydro-benzothiazole-6-carboxylic acid ethyl ester.

¹H-NMR in DMSO: 1.19 ppm (t, 3H); 1.80 ppm (m, 1H); 2.09 ppm (s, 3H); 2.11 ppm (m, 1H); 2.61 ppm (t, 2H); 2.82 ppm (m, 3H), 4.08 ppm (q, 2H).

According to the same procedure, the following compounds are obtained:
2-acetylamino-4,5,6,7-tetrahydro-benzothiazole-6-carboxylic acid methyl ester;
2-acetylamino-4,5,6,7-tetrahydro-benzothiazole-6-carboxylic acid propyl ester;
2-propionylamino-4,5,6,7-tetrahydro-benzothiazole-6-carboxylic acid methyl ester;
2-propionylamino-4,5,6,7-tetrahydro-benzothiazole-6-carboxylic acid ethyl ester; and
2-propionylamino-4,5,6,7-tetrahydro-benzothiazole-6-carboxylic acid propyl ester.

### Example 4

### 2-Acetylamino-4,5,6,7-tetrahydro-benzothiazole-6-carboxylic acid ethyl ester hydrobromide [(VII), R₁=ethyl, R=—NH—CO—CH₃]

A 500 ml reactor equipped with mechanical stirrer, thermometer and condenser was loaded with 200 ml of methylene chloride, 20 g of 4-oxo-cyclohexanecarboxylic acid ethyl ester, 2 g of 48% hydrobromic acid. The resulting clear solution was cooled to 0° C. and dropwise added with 18.88 g of bromine in about 2 hours. Two hours after completion of the addition, 100 ml of water were added and the phases were separated, discarding the aqueous one. 80 ml of water were added and the mixture was neutralized to pH=7-8 with sodium bicarbonate. The organic phase was separated and was concentrated to one third of the original volume, then added with 150 ml of ethanol and 13.95 g of acetyl thiourea to obtain a suspension. Upon reflux, the solid gradually dissolved to obtain a clear solution. After 3 hours the solution was concentrated to small volume to obtain a solid mass, that was crystallized from 200 ml of i-propanol to obtain 15.9 g of solid.

¹H-NMR in DMSO: 1.2 ppm (t, 3H); 1.81 ppm (m, 1H); 2.09 ppm (m, 1H); 2.11 ppm (s, 3H); 2.60 ppm (t, 2H); 2.81 ppm (m, 3H); 4.08 ppm (q, 2H).

According to the same procedure, the following compounds are obtained, as hydrobromide:
2-acetylamino-4,5,6,7-tetrahydro-benzothiazole-6-carboxylic acid methyl ester; and
2-acetylamino-4,5,6,7-tetrahydro-benzothiazole-6-carboxylic acid propyl ester;
2-propionylamino-4,5,6,7-tetrahydro-benzothiazole-6-carboxylic acid methyl ester;
2-propionylamino-4,5,6,7-tetrahydro-benzothiazole-6-carboxylic acid ethyl ester; and
2-propionylamino-4,5,6,7-tetrahydro-benzothiazole-6-carboxylic acid propyl ester.

### Example 5

### 2-Acetylamino-4,5,6,7-tetrahydro-benzothiazole-6-carboxylic acid [(III), R=—NH—CO—CH₃]

A 500 ml reactor equipped with mechanical stirrer, thermometer and condenser was loaded with 200 ml of water, 30 g of 2-acetylamino-4,5,6,7-tetrahydro-benzothiazole-6-carboxylic acid ethyl ester [(VII), R₁=ethyl, R=—NH—CO—CH₃] and 52.2 g of 30% sodium hydroxide, keeping the temperature below 30° C.; during the addition the solid gradually solubilized until complete dissolution. After 2 hours, glacial acetic acid was dropwise added to pH=4.5-5.5; after approx. 1 hour the precipitated white solid was filtered, washed with 70 ml of water and dried to obtain 24.8 g of 2-acetylamino-4,5,6,7-tetrahydro-benzothiazole-6-carboxylic acid.

¹H-NMR in DMSO: 1.75 ppm (m, 1H); 2.09 ppm (s, 3H); 2.11 ppm (m, 1H); 2.58 ppm (m, 3H); 2.78 ppm (m, 2H).

¹³C-NMR in DMSO: 22.48 ppm; 24.72 ppm; 25.04 ppm; 25.5 ppm; 39.37 ppm; 119.77 ppm; 143.4 ppm; 155.27 ppm; 167.99 ppm; 175.69 ppm.

According to the same procedure, 2-propionylamino-4,5,6,7-tetrahydro-benzothiazole-6-carboxylic acid is obtained.

### Example 6

### (R)—N-(6-Amino-4,5,6,7-tetrahydro-benzothiazol-2-yl)-acetamide dihydrochloride, [(II), Ra=—NH—CO—CH₃, R₃=H]

A 500 ml reactor equipped with mechanical stirrer, thermometer and condenser was loaded with 10 g of (R)-2-acetylamino-4,5,6,7-tetrahydro-benzothiazole-6-carboxylic acid [(III), R=—NH—CO—CH₃] suspended in 146 ml of N,N-dimethylformamide, and 4.63 g of triethylamine were added. After that, a solution consisting of 12.57 g of diphenylphosphoryl azide (DPPA) dissolved in 10 ml N,N-dimethylformamide was dropped therein in 2 hours. The reaction mixture gradually solubilized during the addition until complete dissolution. After 5 hours the reaction solution was dropped in 1.3 liters of an aqueous solution containing 14 ml of 37% hydrochloric acid, at 60° C. The mixture was left to cool, then extracted twice with 200 ml of methylene chloride, discarding the organic phase. The aqueous phase was concentrated to a residue, that was crystallized from i-propanol-water to obtain 4.5 g of a white solid.

¹H-NMR in DMSO: 1.91 ppm (m, 1H); 2.17 ppm (s, 3H); 2.19 ppm (m, 1H); 2.73 pm (m, 3H); 3.07 ppm (dd, 1H); 3.49 ppm (s, broad, 1H); 8.39 ppm (s, broad, 2H).

¹³C-NMR in DMSO: 22.50 ppm; 23.64 ppm; 26.49 ppm; 26.66 ppm; 46.56 ppm; 117.39 ppm; 142.89 ppm; 156.06 ppm; 168.28 ppm.

### Example 7

### (R)-(2-Acetylamino-4,5,6,7-tetrahydro-benzothiazol-6-yl)-carbamic acid methyl ester hydrochloride [(II), Ra=—NH—CO—CH₃, R₃=—CO—O—CH₃]

A 500 ml reactor equipped with mechanical stirrer, thermometer and condenser is loaded with 5 g of (R)-2-acetylamino-4,5,6,7-tetrahydro-benzothiazole-6-carboxylic acid [(III), R=—NH—CO—CH₃] suspended in 80 ml of N,N-dimethylformamide; then 2.32 g of triethylamine are added. A solution consisting of 6.3 g of diphenylphosphoryl azide (DPPA) dissolved in 7 ml N,N-dimethylformamide is dropped therein in 2 hours. The reaction mixture gradually solubilizes during the addition until complete dissolution. After 6 hours the reaction solution is dropped in 1 liter of a methanol solution containing 8 ml of 37% hydrochloric acid at 60° C. The mixture is left to cool, then extracted twice with 100 ml of methylene chloride, discarding the organic phase. The aqueous phase is concentrated to a residue that is crystallized from i-propanol-water to obtain 3.6 g of a white solid.

### Example 8

### Resolution of (R) 2-acetylamino-4,5,6,7-tetrahydro-benzothiazole-6-carboxylic acid [(III), R=—NH—CO—CH₃]

A 1 liter reactor equipped with mechanical stirrer, thermometer and condenser are loaded with 50 g of 2-acetylamino-4,5,6,7-tetrahydro-benzothiazole-6-carboxylic acid [(III), R=—NH—CO—CH₃] suspended in 250 ml of methanol and 50 ml of water. The mixture is heated until dissolution, added with 37.3 g of (R)-(+)-α-methylbenzylamine, cooled to 25° C., and the precipitated product is filtered off, washed with methanol and dried to obtain 42.8 g of a solid. This is suspended in 250 ml of methanol and 50 ml of water, heated to dissolution for 1 hour and cooled to room temperature. The suspended solid is filtered, washed with methanol and dried to obtain 32.3 g of (R)-2-acetylamino-4,5,6,7-tetrahydro-benzothiazole-6-carboxylic acid, having enantiomeric purity >99.5%.

By proceeding analogously the compound (R)-2-propionyllamino-4,5,6,7-tetrahydro-benzothiazole-6-carboxylic acid can be prepared.

### Example 9

### (R)—N-{6-[3-(2-acetylamino-4,5,6,7-tetrahydro-benzothiazol-6-yl)-ureido]-4,5,6,7-tetrahydro-benzothiazol-2-yl}-acetamide, [(IIIc), Y═NH₂; R=—NH—CO—CH₃]

A 500 ml reactor equipped with mechanical stirrer, thermometer and condenser was loaded with 10 g of (R)-2-acetylamino-4,5,6,7-tetrahydro-benzothiazole-6-carboxylic acid [(III), R=—NH—CO—CH₃] suspended in 146 ml of N,N-dimethylformamide and 4.65 g of triethylamine were added. A solution consisting of 12.52 g of diphenylphosphoryl azide (DPPA) dissolved in 10 ml N,N-dimethylformamide was dropped therein in 2 hours. The reaction mixture gradually solubilized during the addition until complete dissolution. After 5 hours, the reaction mixture was dropped in 1.3 liters of an aqueous solution at 60° C. The mixture was left to cool, the separated solid was filtered, washing twice with 50 ml of water to obtain 5.9 g of a white solid.

¹H-NMR in DMSO: 1.72 ppm (m, 1H); 1.86 ppm (m, 1H); 2.07 ppm (s, 3H); 2.4 ppm (dd, 1H); 2.59 ppm (m, 2H); 2.8 ppm (dd, 1H); 3.93 ppm (m, 1H), 5.96 ppm (d, 1H), 11.84 ppm (s, 1H).

¹³C-NMR in DMSO: 22.30 ppm; 23.74 ppm; 26.55 ppm; 26.59 ppm; 44.36 ppm; 118.42 ppm; 144.02 ppm; 156.13 ppm; 157.98 ppm, 169.18 ppm.

### Example 10

### (R)—N-(6-Propionylamino-4,5,6,7-tetrahydro-benzothiazol-2-yl)-amine; [(VI) Ra=NH₂]

A 1 liter reactor equipped with mechanical stirrer, thermometer and condenser is loaded with under nitrogen 43.7 g of (R)—N-(6-amino-4,5,6,7-tetrahydro-benzothiazol-2-yl)-amine and 220 ml of methyl ethyl ketone (MEK). is heated a 28-32° C. and approx. 33.6 g of propionic anhydride are dropped therein in 2 hours keeping the temperature at about 28-32° C. The solution is cooled to about 0-5° C. and 109 g of 10% aqueous NaOH are added. The aqueous phase is separated; the organic phase is diluted with 60 ml of toluene and concentrated under vacuum at about 40-45° C. Under these conditions, the product starts to crystallize. The suspension is cooled to 0-5° C. and left under stirring for an hour. The precipitate is filtered with suction and washed with 10 ml of toluene.

54.2 g of (R)N-(6-propionylamino-4,5,6,7-tetrahydro-benzothiazol-2-yl)-amine are obtained.

### Example 11

### (I)— Dexpramipexole Free Base

A 2 liter reactor under nitrogen is loaded with 53.3 g of (R)N-(6-propionylamino-4,5,6,7-tetrahydro-benzothiazol-2-yl)-amine, 33.0 g of 95% sodium borohydride and 260 ml of tetrahydrofuran (THF). A solution of 98.7 g of iodine in 160 ml of THF is dropped therein in about 3 hours, keeping the temperature at approx. 20-25° C. The reaction mixture is kept under stirring for further 2 hours at about 20-25° C. The reaction mixture is poured into a solution of 60.0 g of 37% HCl in 260 ml of water. The mixture is heated to 50-55° C. and left under stirring for an hour. The complete cleavage of the boran-complexes is checked by HPLC. The mixture is added with 250 g of 50% aqueous NaOH, keeping the temperature at about 20-25° C. After that, 315 ml of toluene are added and the mixture is heated to about 30-35° C. Stirring is interrupted and the two phases are separated. The organic phase are washed, concentrated to a residue and dissolved in 420 ml of ethyl acetate.

The solution is concentrated under vacuum at a temperature below 50° C. to about 150 ml volume. The resulting suspension is refluxed, then cooled to about 10-15° C., stirred for further 1-2 hours, then filtered with suction and the precipitate is washed twice with 30 ml of ethyl acetate. The product is dried under vacuum at 40° C. 32 g of (R)-2-amino-6-propylamino-4,5,6,7-tetrahydrobenzothiazole are obtained. The crystalline product is characterised by an XRPD spectrum as shown in FIG. 1, wherein the most intense peaks fall at 9.66, 10.77, 11.10, 12.99, 14.55, 16.56, 17.19, 17.94, 18.72, 21.42, 21.84, 23.49, 24.21, 27.00 and 28.23±0.2° in 2θ.

### Example 12

### Isopropyl (R)-(2-acetylamino-4,5,6,7-tetrahydro-benzothiazol-6-yl)-carbamate [(II), Ra=-NH—CO—CH₃, R₃=—CO—O—C₃H₇]

A 2000 ml reactor equipped with mechanical stirrer, thermometer and condenser is loaded with 100 g of (R)-2-acetylamino-4,5,6,7-tetrahydro-benzothiazole-6-carboxylic acid [(III), R=—NH—CO—CH₃] of 97% enantiomeric purity, suspended in 700 ml of isopropanol; 84.16 g of triethylamine are added. The mixture is refluxed (about 80° C.) and a solution consisting of 120.42 g of diphenylphosphoryl azide (DPPA) is dropped therein in 2 hours. After 2 hours, the reaction mixture is cooled to 20-30° C. and added with

500 ml of water and 1.6 g of sodium hydroxide. Isopropanol is distilled off under vacuum, then 400 ml of ethyl acetate are added. The mixture is refluxed for 15 minutes, then the hot suspension is filtered through Celite. The solution is cooled to 20-30° C. and added with of 1800 ml of water. The phases are separated and the organic phase concentrated to dryness. The residue is taken up with 200 ml of acetonitrile. The suspension is heated at 50° C. for 1 hour, then cooled to 20° C. and filtered to obtain 75 g of isopropyl (R)-(2-acetylamino-4,5,6,7-tetrahydro-benzothiazol-6-yl)-carbamate, with 97% enantiomeric purity.

### Example 13

### Dexpramipexole Monohydrate Dihydrochloride Salt

A 500 ml reactor equipped with mechanical stirrer, thermometer and condenser is loaded with 18.5 g of (R)-2-amino-6-propylamino-4,5,6,7-tetrahydrobenzothiazole and 74 ml of isopropanol. The suspension is warmed up to 40° C., then 17.6 g of HCl (36%) and 2.1 ml of water are added. The mixture is now warmed to reflux temperature and 0.95 ml of water are dropped, in order to obtain a clear solution. 120 ml of isopropanol are now dropped in about 2 hours, in way of crystallizing the product. The resulting suspension is maintained at reflux temperature for 1 hour, cooled down to 20° C. in 4 hours, then filtered. 21.6 g of Dexpramipexole monohydrate dihydrochloride salt are obtained, with an enantiomeric purity typically of at least about 99.9%, in particular equal to or higher than 99.95%. The obtained crystalline product is characterized by an XRPD spectrum as shown in FIG. 2, wherein the most intense peaks fall at 6.48, 12.06, 12.93, 13.29, 13.92, 15.15, 16.95, 17.25, 17.91, 19.47, 21.36, 24.21, 24.75, 24.99, 25.59 and 28.44±0.2° in 2θ; an IR spectrum as shown in FIG. 3, showing the most intense peaks at 3411, 2945, 2742, 2440, 1651, 1627, 1432, 1362, 1218, 1070, 1042, 982, 760 e 714 cm⁻¹; and has a water content ranging between about 5 and about 7% w/w, typically around 6% w/w.

1. Process for the preparation of a compound of formula (I), in a solid state thereof, or a salt thereof, as the single (R) enantiomer, 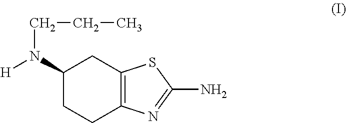
comprising alkylating a compound of formula (II), as the single (R) enantiomer, 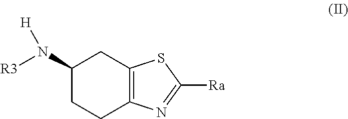
wherein Ra is a free or protected amino group, R₃ is hydrogen or a R₄—O—CO— group, wherein R₄ is a straight or branched C₁-C₄ alkyl, to obtain a compound of formula (V) 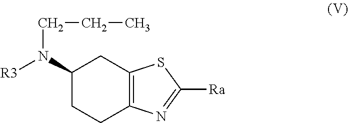
wherein Ra and R₃ are as defined above, and, if necessary, removing the primary amino-protecting group and/or the R₄—O—CO— group from the secondary amino group, and/or, if the case, converting a compound of formula (I) into a salt thereof, and/or, if the case, converting a salt of a compound of formula (I) into the free compound,
characterized in that:
a) a compound of formula (II), as single (R) enantiomer, or a salt thereof, 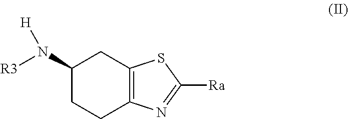
wherein Ra is a protected amino group and R₃ is as defined above, is prepared by rearrangement of a compound of formula (III), or a salt thereof, as single (R) enantiomer, 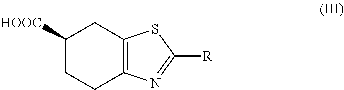
wherein R is a protected amino group; via formation of isocyanate, and subsequent addition of a nucleophilic solvent or subsequent quenching in water in the presence of an acid; or
b) a compound of formula (II), as single (R) enantiomer, or a salt thereof, 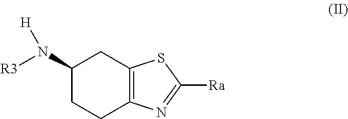
wherein Ra is a free amino group and R₃ is hydrogen; is prepared by rearrangement of a compound of formula (III), or a salt thereof, as single (R) enantiomer, as defined above; via formation of isocyanate, and subsequent addition of water, to obtain a compound of formula (IV) 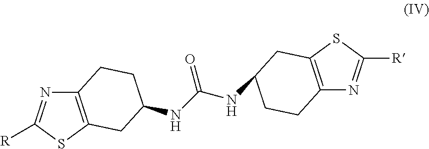
wherein R′ has the same meaning as R above, and subsequent hydrolysis.

2. A process according to claim 1, variant a) wherein quenching in water in the presence of an acidic agent affords a compound of formula (II), as defined in claim 1, wherein R₃ is hydrogen.

3. A process according to claim 1, variant a), wherein the nucleophilic solvent is a C₁-C₄ alkanol, to obtain a compound of formula (II), as defined in claim 1, wherein R₃ is a R₄—O—CO— group, wherein R₄ is as defined in claim 1.

4. A process according to claim 1, variant a), wherein the rearrangement reaction is carried out according to Curtius in a nucleophilic solvent, via formation of a compound of formula of formula (IIIa) 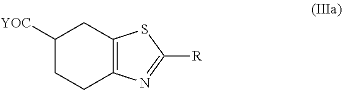
in which Y is N₃;
and of a compound of formula (IIId) 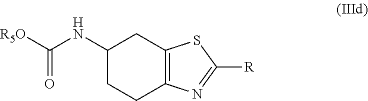
wherein R₅ is a straight or branched C₁-C₄ alkyl group, without recovery of the intermediates.

5. A process according to claim 1, wherein the rearrangement takes place via formation of a isocyanate of formula (IIIc) 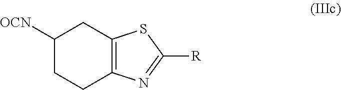
in which R is a protected amino group, and subsequent addition of a nucleophilic solvent or subsequent quenching in water in the presence of an acidic agent.

6. A process according to claim 1, wherein the compound of formula (I) is R)-2-amino-6-n-propylamino-4,5,6,7-tetrahydrobenzothiazole free base, in crystalline form.

7. A process according to claim 1, wherein the compound of formula (I) is (R)-2-amino-6-n-propylamino-4,5,6,7-tetrahydrobenzothiazole free base, in crystalline form, characterized by an XRPD spectrum as shown in FIG. 1, wherein the most intense peaks fall at 9.66, 10.77, 11.10, 12.99, 14.55, 16.56, 17.19, 17.94, 18.72, 21.42, 21.84, 23.49, 24.21, 27.00 and 28.23±0.2° in 2q.

8. A process according to claim 1, wherein a salt of a compound of formula (I) is (R)-2-amino-6-n-propylamino-4,5,6,7-tetrahydrobenzothiazole monohydrate dihydrocloride salt in crystalline form.

9. A process according to claim 1, wherein a salt of a compound of formula (I) is (R)-2-amino-6-n-propylamino-4,5,6,7-tetrahydrobenzothiazole monohydrate dihydrocloride salt, in crystalline form, characterised by an XRPD spectrum as shown in FIG. 2, wherein the most intense peaks fall at 6.48, 12.06, 12.93, 13.29, 13.92, 15.15, 16.95, 17.25, 17.91, 19.47, 21.36, 24.21, 24.75, 24.99, 25.59 and 28.44±0.2° in 2q; and an IR spectrum as shown in FIG. 3 showing the most intense peaks at 3411, 2945, 2742, 2440, 1651, 1627, 1432, 1362, 1218, 1070, 1042, 982, 760 e 714 cm⁻¹.

10. A process according to claim 9, wherein said salt of a compound of formula (I) has an enantiomeric purity of at least about 99.9%.

11. (R)-2-amino-6-n-propylamino-4,5,6,7-tetrahydrobenzothiazole free base, in crystalline form.

12. (R)-2-amino-6-n-propylamino-4,5,6,7-tetrahydrobenzothiazole free base, in crystalline form, characterized by an XRPD spectrum as shown in FIG. 1, wherein the most intense peaks fall at 9.66, 10.77, 11.10, 12.99, 14.55, 16.56, 17.19, 17.94, 18.72, 21.42, 21.84, 23.49, 24.21, 27.00 and 28.23±0.2° in 2q.

13. (R)-2-amino-6-n-propylamino-4,5,6,7-tetrahydrobenzothiazole monohydrate dihydrocloride salt, in crystalline form.

14. (R)-2-amino-6-n-propylamino-4,5,6,7-tetrahydrobenzothiazole monohydrate dihydrocloride salt, in crystalline form, characterised by an XRPD spectrum as shown in FIG. 2, wherein the most intense peaks fall at 6.48, 12.06, 12.93, 13.29, 13.92, 15.15, 16.95, 17.25, 17.91, 19.47, 21.36, 24.21, 24.75, 24.99, 25.59 and 28.44±0.2° in 2q; and an IR spectrum as shown in FIG. 3, showing the most intense peaks at 3411, 2945, 2742, 2440, 1651, 1627, 1432, 1362, 1218, 1070, 1042, 982, 760 e 714 cm⁻¹.

15. (R)-2-amino-6-n-propylamino-4,5,6,7-tetrahydrobenzothiazole monohydrate dihydrocloride salt, in crystalline form, as defined in claim 13, having a water content ranging between about 5 and about 7% w/w.

16. (R)-2-amino-6-n-propylamino-4,5,6,7-tetrahydrobenzothiazole monohydrate dihydrocloride salt, in crystalline form, as defined in claim 13, with an enantiomeric purity of at least about 99.9%.
